(19) United States
(12) Patent Application Publication
(10) Pub. No.: US 2022/0356155 A1
(43) Pub. Date: Nov. 10, 2022

(54) FUEL HIGH TEMPERATURE ANTIOXIDANT ADDITIVE

(71) Applicant: ExxonMobil Research and Engineering Company, Annandale, NJ (US)

(72) Inventors: David J. Abdallah, Moorestown, NJ (US); Krystal B. Wrigley, Asbury, NJ (US); Zsolt Lengyel, Newtown, PA (US)

(21) Appl. No.: 17/245,219

(22) Filed: Apr. 30, 2021

(51) Int. Cl. C07D 211/10; C10L 1/232

(52) U.S. Cl. CPC C07D 211/10; C10L 1/232; C10L 2230/081; C10L 2270/02

(57) ABSTRACT

High temperature antioxidant additives and methods for improving a liquid fuel composition's thermal oxidative stability are disclosed. Also provided are liquid fuel compositions including a liquid fuel and the high temperature antioxidant additives. The high temperature antioxidant additive may include an alkylpiperidin-1-yl alkylcarbamate.

### FIELD

This disclosure relates to high temperature antioxidant additives for liquid fuels, and, more particularly, relate to high temperature antioxidant additives with alkylpiperidin-1-yl alkylcarbamate functionality and methods that improve a liquid fuel's thermal oxidative stability.

### BACKGROUND

Operation of an internal combustion engine can lead to deposits in the fuel system. The deposits can adversely impact engine performance, potentially resulting in fuel system component malfunction or failure. For instance, the deposits can restrict the flow of air and fuel entering the combustion chamber, which can cause stalling and hesitation. One contributor to fuel system deposits is from fuel degradation caused by oxidation reactions between molecular oxygen and the fuel. This process is accelerated with higher temperatures. To achieve better combustion and reduced emissions, modern engine designs have trended toward higher fuel system operating temperatures and pressures, thus subjecting fuels to higher thermal loads than has been typical in the past. However, the increased thermal loads can lead to increased fuel oxidation and, thus, increased deposits.

One technique that has been used for fuel-system deposit control has been to use detergents. However, detergents typically do not work across the entire fuel system and may be designed to target specific components within the fuel system, e.g., carburetor detergents, intake valve detergents, valve stem deposit fluidizers, port fuel injector detergents, and direct fuel injector detergents among others. In some instances, a detergent targeting a specific component can cause deposits in other components of the fuel system. For instance, high levels of carburetor detergents can increase piston ring belt deposits and intake valve deposits, while intake valve detergents that can clean the tops of valve tulips can create sticky valve stem deposits. Additionally, these detergents and the fluidizers that often accompany them are typically not conducive to combusting and tend to contribute to combustion chamber deposits, which are known to lead to octane rating increase, combustion chamber deposit interference, disturbance of the air-fuel mixture formation, and/or increased regulated emissions. In addition, while detergents are designed to address the deposits that can result from oxidation, they are not designed to stop oxidation from occurring. While antioxidant additives have been included in fuels, they are designed to combat oxidation and preserve fuel stability at ambient storage conditions rather than engine operating temperatures. At increased temperatures, these antioxidants can degrade and lead to fuel system deposits.

There is a need for antioxidants that are effective at elevated temperatures to control fuel oxidation that results in fuel system deposits and improve the oxidative stability of a fuel composition.

### SUMMARY

Disclosed herein are high temperature antioxidant additives and liquid fuel compositions including such additives. The inventive liquid fuel compositions comprise a liquid fuel and one or more high temperature antioxidant additives. The high temperature antioxidant additive may comprise alkylpiperidin-1-yl group attached to alkylcarbamic acid group to make alkylpiperidin-1-yl alkylcarbamate.

Further disclosed herein is another exemplary liquid fuel composition. The exemplary liquid fuel composition may comprise a liquid fuel in an amount of about 99 vol. % or greater and a high temperature antioxidant additive. The high temperature antioxidant additive may comprise alkylpiperidin-1-yl alkylcarbamate. The inventive high temperature antioxidant additive disclosed herein is of the following formula:

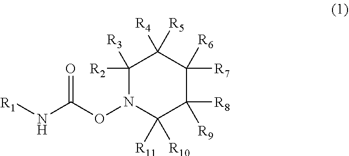

wherein R₁ is a hydrogen, an alkyl group, an alkenyl group, a heteroatom substituted alkyl group, or a heteroatom substituted alkenyl group, R₂, R₃, R₄, R₅, R₆, R₇, R₈, R₉, R₁₀, and R₁₁are individually selected from hydrogen, alkyl groups, alkenyl groups, heteroatom substituted alkyl groups, heteroatom substituted alkenyl groups or a hydroxyl group.

Further disclosed herein is a method for improving thermal oxidative stability of a liquid fuel at high thermal loads, wherein the method includes providing the inventive liquid fuel compositions to an internal combustion engine and combusting in the internal combustion engine the liquid fuel composition comprising a liquid fuel and a the inventive high temperature antioxidant additives disclosed herein. In one form, the high temperature antioxidant additive comprises an alkylpiperidin-1-yl alkylcarbamate.

### BRIEF DESCRIPTION OF THE DRAWINGS

These drawings illustrate certain aspects of the present invention and should not be used to limit or define the invention.

FIG. 1 illustrates an autoxidative free radical chain reaction process for a fuel oxidative degradation.

FIG. 2 is a chart showing the oxidation induction period for a ULSD with 100 ppm of a high temperature antioxidant additive and the oxidation induction period for a comparative sample without the antioxidant additive. The high temperature antioxidant additive was 2,2,6,6-tetramethylpiperidin-1-yl (2-ethylhexyl)carbamate (Formula 2 with R₁ equal to 2-ethylhexyl).

FIG. 3 is a chart showing the oxidation induction period for a ULSD with 100 ppm of a high temperature antioxidant additive and the oxidation induction period for a comparative sample without the antioxidant additive. The high temperature antioxidant additive was 2,2,6,6-tetramethylpiperidin-1-yl octadecylcarbamate (Formula 2 with R₁ equal to octadecyl).

FIG. 4 is a chart showing the oxidation induction period for a ULSD with 100 ppm of a high temperature antioxidant additive and the oxidation induction period for a comparative sample without the antioxidant additive. The high temperature antioxidant additive was 2,2,6,6-tetramethylpiperidin-1-yl octylcarbamate (Formula 2 with R1 equal to octyl).

FIG. 5 is a chart showing the oxidation induction periods for a ULSD with 100 ppm of known storage stability antioxidant additives and the oxidation induction period for a comparative sample without the additives.

### DETAILED DESCRIPTION

All numerical values within the detailed description and the claims herein are modified by “about” or “approximately” the indicated value, and take into account experimental error and variations that would be expected by a person having ordinary skill in the art.

This disclosure relates to high temperature antioxidant additives for liquid fuels, and, more particularly, embodiments relate to high temperature antioxidant additives with alkylpiperidin-1-yl alkylcarbamate functionality and methods that improve a liquid fuel composition's thermal oxidative stability. As used herein, the antioxidant additives are referred to as “high temperature” antioxidant additives because the antioxidant additives improve a liquid fuel composition's thermal oxidative stability. Embodiments disclose an antioxidant additive that includes alkylpiperidin-1-yl alkylcarbamate functionality to improve the thermal oxidative stability of a liquid fuel composition. Thermal oxidative stability is measured in terms of the liquid fuel composition's tendency to form deposits in the fuel system, including fuel lines, heat exchangers and nozzles of jet engines as well as on the intake valves, ports, fuel injectors, and combustion chamber surfaces of gasoline and diesel engines. By operation improvement of the thermal oxidative stability, the antioxidant additives may not only help with fuel storage stability but also provide benefits to the liquid fuel composition at engine operating temperatures.

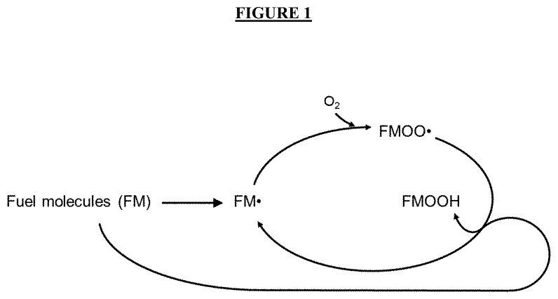

During heating of a liquid fuel composition, for example, in operation of an engine, fuel oxidative degradation proceeds through an autoxidative free radical chain reaction process. An example reaction scheme for fuel oxidative degradation is provided in FIG. 1. The fuel molecules (shown as FM) present in the liquid fuel composition break down into free radicals (shown as FM·). Propagation reactions may then occur in which the free radicals combine with oxygen to form peroxide radicals (shown as FMOO·) which abstract hydrogen from another fuel molecule, or within the same fuel molecule, to form a new FM and a hydroperoxide. Termination reactions may then occur in which the peroxide radicals are eliminated. The termination reactions include reaction of the peroxide radicals with additional fuel molecule radicals to form peroxides. Hydroperoxides formed from the chain reaction are inherently unstable to heat and can readily decompose to yield additional free radicals (e.g., FM· and OH·), which continue to initiate additional chain reactions and additional hydroperoxides (shown as FMOOH). Hydroperoxides are a primary product of autoxidation and therefore may be considered the main initiators in thermal oxidation. Hydroperoxides, and their decomposition products are ultimately responsible for the changes in molecular structure and fuel system deposits. Conventional antioxidants produce hydroperoxides that stop the chain reaction at storage temperatures but can decompose to produce free radicals when heated. However, the high temperature antioxidant additive disclosed herein comprising an alkylpiperidin-1-yl ring attached to an alkylcarbamate functionality should delay the oxidation induction period of the liquid fuel composition. As the oxidation induction period is delayed less peroxide radicals are generated, leading to less hydroperoxides and ultimately less deposits. In other words, the antioxidant additive may be considered to block fuel degradation pathways at high temperatures.

There may be several potential advantages to the compositions and methods disclosed herein, only some of which may be alluded to in the present disclosure. One of the many potential advantages of the compositions and methods is that the antioxidant additive should extend the oxidation induction period of the liquid fuel composition. The oxidation induction period is an initial slow stage of fuel oxidation after which the oxidation reaction accelerates. By extending the oxidation induction period, fuel oxidation in the fuel system that leads to deposits may be reduced or potentially avoided. In some embodiments, the oxidation induction period may be extended to a timeframe that is longer than the liquid fuel composition will spend at elevated temperatures in the fuel system components.

Suitable antioxidant additives may include an alkylpiperidin-1-yl alkylcarbamate group. Additional substituents may also be present on the aliphatic ring of the antioxidant additive, including, but not limited to, alkyl groups, alkenyl groups, hetero-atom substituted alkyl groups, or hetero-atom substituted alkenyl groups.

High temperature alkylpiperidin-1-yl alkylcarbamate antioxidants may be made by the reaction of amines with an acyl transfer reagent, 4-nitrophenyl (2,2,6,6-tetramethylpiperidin-1-yl) carbonate (NPTC). NPTC may be prepared in two steps from (2,2,6,6-Tetramethylpiperidin-1-yl)oxyl, commonly known as TEMPO, via reduction with sodium ascorbate and acylation with p-nitrophenyl chloroformate. The 2,2,6,6-tetramethylpiperidin-1-yloxycarbonyl (Tempoc) has been used as a protecting group for amines. Synthetic details can be found in the Journal Organic Letters 2018 20 (21), 6760-6764 authored by Joseph R. Lizza et al., and herein incorporated by reference in its entirety. Both primary and secondary amines are converted to the Tempoc derivatives using NPTC (1.2 equiv), amine (1 equiv), and triethylamine (3 equiv), in dimethylformamide (0.5M, room temperature, 12 hours). Other acyl transfer reagent may be made from stable aminoxyl radical compounds other than TEMPO such as 4-hydroxy-2,2,6,6-tetramethylpiperidin-1-oxyl, TEMPO methacrylate, poly(TEMPO methacrylate).

Examples of suitable antioxidant additives may include, but are not limited to, an alkylpiperidin-1-yl alkylcarbamate of Formula (1) as follows:

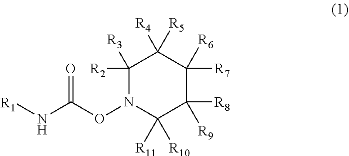

wherein R₁ is a hydrogen, an alkyl group, an alkenyl group, a heteroatom substituted alkyl group, or a heteroatom substituted alkenyl group, R₂, R₃, R₄, R₅, R₆, R₇, R₈, R₉, R₁₀, and R₁₁ are individually selected from hydrogen, alkyl groups, alkenyl groups, heteroatom substituted alkyl groups, heteroatom substituted alkenyl groups or a hydroxyl group.

R₁ is preferably an alkyl groups of the aliphatic series. The aliphatic series includes those that contain no rings and have the general formula CnH2n+1 (e.g. pentyl, hexyl, heptyl, octyl) or contain one ring and have the general formula CnH2n−1 (e.g. cyclooctyl and cyclohexyl). R1 is preferably a higher alkyl group, wherein n is equal or greater than 5 to provide fuel solubility. Side chains with five or more carbons may provide the additive the solubility required for hydrocarbon fuels. More preferably R1 is a substituted alkyl group, for example, but not limited to, 3-methylpentyl, 2-methylpentyl, 3-ethylpentyl, 2,3-dimethylhexyl, iso-pentyl, sec-pentyl, 2-ethylhexyl.

Yet another example of a suitable antioxidant additive may include an alkylpiperidin-1-yl alkylcarbamate of Formula 1, wherein R₁ is alkyl group and R₂, R₃, R₁₀, and R₁₁ are each methyl and R₄, R₅, R₆, R₇, R₈, and R₉ are each a Hydrogen group. Such an additive is commonly referred to as 2,2,6,6-tetramethylpiperidin-1-yl alkylcarbamate and has the following structure:

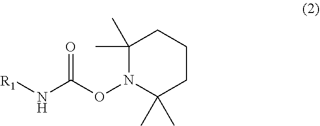

As previously described, the high temperature antioxidant additive comprising alkylpiperidin-1-yl alkylcarbamate can be used to improve a liquid fuel composition's thermal oxidative stability. The high temperature antioxidant additive may be included in the liquid fuel composition in any suitable amount as desired for improving thermal oxidative stability. In some embodiments, the high temperature antioxidant composition can be present in the liquid fuel composition in an effective amount with an effective amount ranging from about 0.1 parts per million (“ppm”) to about 500 ppm and, more particularly, ranging from about 1 ppm to about 200 ppm. In some embodiments, the high temperature antioxidant additive may be present in the liquid fuel composition in an amount of about 0.1 ppm, about 0.5 ppm, about 1 ppm, about 5 ppm, about 10 ppm, about 25 ppm, about 50 ppm, about 100 ppm, about 200 ppm, about 300 ppm, about 400 ppm, or about 500 ppm. One of ordinary skill in the art with the benefit of this disclosure should be able to select an appropriate amount of the high temperature antioxidant additive based on a number of factors, including, but not limited to, fuel system operating conditions, the particular alkyl group on the alkylpiperidin-1-yl alkylcarbamate, and the liquid fuel's hydrocarbon components, among others.

In some embodiments, the high temperature antioxidant additive comprising alkylpiperidin-1-yl alkylcarbamate may be included in a liquid fuel composition to extend an oxidation induction period of the liquid fuel composition, which should result in improved thermal stability. The oxidation induction period may be extended as compared to the liquid fuel composition without the high temperature antioxidant additive, for example, from about 10% to 200%, or longer than the fuel without the additive. In some embodiments, the oxidation induction period may be extended as compared to the liquid fuel composition without the high temperature antioxidant additive for period of about 200 seconds, about 500 seconds, about 1,000 seconds, about 2,000 seconds, about 5,000 seconds, about 10,000 seconds, or even longer. The oxidation induction period is an initial slow stage of fuel oxidation after which the oxidation reaction accelerates. As used herein, the oxidation induction period is determined using ASTM D 525 (pressure vessel method) or the PetroOXY automatic oxidation stability tester using a test method based on ASTM D 7545 Standard Test Method for Oxidation Stability of Middle Distillate Fuels—Rapid Small Scale Oxidation Test (RSSOT) or ASTM D 7245 Oxidation Stability of Spark Ignition Fuel—Rapid Small Scale Oxidation Test (RSSOT). In the pressure vessel method, a 50 mL sample is oxidized in a pressure vessel initially filled at 15 to 25° C. with oxygen pressure at 690 to 705 kPa and heated at a temperature between 98 and 102° C. The pressure is recorded continuously or read at stated intervals until the breakpoint is reached. The time required for the sample to reach this point is the observed induction period at the temperature of test, from which the induction period at 100° C. can be calculated. In the RSSOT test methods, a 5 mL sample of the liquid fuel composition is combined with oxygen starting at a pressure of 500 kPa for motor gasoline or 700 kPa for diesel in a small, hermetically seal test chamber and heated to a test temperature. Pressure increases as the temperature of the vessel is increased from the volatilization of the light components of the fuel. Pressure is monitored over time. End of test is where a 10% drop in pressure from the maximum vessel pressure is measured. Tests temperatures are chosen that reflect relevant fuel end use temperatures in fuel systems. It has been determined that the time needed to achieve a pressure drop is directly related to induction period of the fuel composition and, thus, the thermal oxidation stability of the fuel composition. The test temperature for diesel fuel is 180° C. corresponding to a severe condition a fuel would experience in a diesel fuel injector tip. The test temperature for motor gasoline is 155° C. corresponding to a severe condition a fuel would experience in a gasoline fuel injector tip. Lower temperatures were used when the fuel composition was not able to obtain the severe conditions such as in for biodiesel testing.

In some embodiments, the high temperature antioxidant additive comprising alkylpiperidin-1-yl alkylcarbamate may be included in a liquid fuel composition to extend the maximum temperature of the liquid fuel composition for the same induction period. The maximum temperature may be extended as compared to the liquid fuel composition without the high temperature antioxidant additive, for example, from about 5° C. to 50° C. or higher than the fuel without the additive. In some embodiments, the maximum temperature may be extended as compared to the liquid fuel composition without the high temperature antioxidant additive for about an additional 10° C., about 20° C., about 100° C., or even higher.

In some embodiments, the inventive fuel compositions including an effective amount of the inventive high temperature antioxidant additive provide for a thermal oxidative stability as measured by D7545 that is improved by from 10% to 200%, or 20% to 150%, or 40% to 120%, or 60% to 100% compared to the same liquid fuel composition, but not including the effective amount of the inventive antioxidant additive disclosed herein.

In some embodiments, the high temperature antioxidant additive may be introduced into a fuel system of an internal combustion engine. In some embodiments, the high temperature antioxidant combination may be combined with the liquid fuel composition in the internal combustion engine. In some embodiments, the high temperature antioxidant composition may be introduced into the internal combustion engine as a component of the liquid fuel composition. In a combustion chamber of the internal combustion engine, the liquid fuel composition may be burned. Suitable internal combustion engines may include, but are not limited to, rotary, turbine, rocket, spark ignition, compression ignition, 2-stroke, or 4-stroke engines. In some embodiments, the internal combustion engines include marine engines, aviation piston and turbine engines, aviation supersonic turbine engines, rocket engine, diesel engines, and automobile and truck engines. In some embodiments, the internal combustion engine may comprise a direct injection engine. In some embodiments, the internal combustion engine may comprise a supersonic turbine engine. In some embodiments, the internal combustion engine may comprise a high pressure common-rail direct fuel injection engine.

In addition to the high temperature antioxidant additive, the liquid fuel composition may further include a liquid fuel. The liquid fuel may include, but are not limited to, motor gasoline, aviation fuel, supersonic fuel, rocket fuel, marine fuel, and diesel fuel. Combinations of different liquid fuels may also be used. Motor gasoline includes a complex mixture of relatively volatile hydrocarbons blended to form a fuel suitable for use in spark-ignition engines. Motor gasoline, as defined in ASTM Specification D4814, is characterized as having a boiling range of 50° C. to 70° C. at the 10-percent recovery point to 185° C. to 190° C. at the 90-percent recovery point. The diesel fuel can be a petroleum distillate as defined by ASTM specification D975. The aviation fuels can be a petroleum distillate as defined by ASTM specification D1655. The supersonic fuel can be a compound mixture composed primarily of hydrocarbons; including alkanes, cycloalkanes, alkylbenzenes, indanes/tetralins, and naphthalenes. As used herein, a supersonic fuel is a fuel that meets the specification for propellant, rocket grade kerosene (either RP-1 or RP-2) in MIL-DTL-25576, dated Apr. 14, 2006. Supersonic fuels are typically capable of standing up to higher heats (without undesirable breakdown) from air friction on the aircraft at speeds greater than the speed of sound. Fuel that breaks down can potentially clog the fuel pipes on its way to the burner. Additional examples of suitable liquid fuels may include, but are not limited to, an alcohol, an ether, a nitroalkane, an ester of a vegetable oil, or combinations thereof. In some embodiments, the other fuels may include, but are not limited to, methanol, ethanol, diethyl ether, methyl t-butyl ether, nitromethane, and methyl esters of vegetable oils such as the methyl ester of rapeseed oil. In some embodiments, the liquid fuel may include a mixture of a motor gasoline and ethanol or a mixture of a diesel fuel and a biodiesel fuel, such as an ester of a vegetable oil.

The diesel fuel compositions of the present disclosure may be a renewable fuel such as a biofuel composition or biodiesel composition. The diesel fuel compositions disclosed herein may optionally include a first generation biodiesel. First generation biodiesel contains esters of, for example, vegetable oils, animal fats and used cooking fats. This form of biodiesel may be obtained by transesterification of oils, for example rapeseed oil, soybean oil, safflower oil, palm oil, corn oil, peanut oil, cotton seed oil, tallow, coconut oil, physic nut oil (Jatropha), sunflower seed oil, and used cooking oils.

The diesel fuel compositions disclosed herein may optionally include a second generation biodiesel. Second generation biodiesel is derived from renewable resources such as vegetable oils and animal fats and processed, often in the refinery, often using hydroprocessing such as the H-Bio process developed by Petrobras. Second generation biodiesel may be similar in properties and quality to petroleum based fuel oil streams, for example renewable diesel produced from vegetable oils, animal fats etc. and marketed by ConocoPhillips as Renewable Diesel and by Neste as NExBTL. These fuels are also referred to as Hydroproccessed Vegetable Oil (HVO) as they are produced by the hydrogenation of vegetable oils or animal fats.

The diesel fuel compositions disclosed herein may optionally include a third generation biodiesel. Third generation biodiesel utilizes gasification and Fischer-Tropsch technology including those described as BTL (biomass-to-liquid) fuels. Third generation biodiesel does not differ widely from some second generation biodiesel, but aims to exploit the whole plant (biomass) and thereby widens the feedstock base.

The diesel fuel compositions disclosed herein may also contain blends of any or all of the above first, second and third generation biodiesels. The liquid fuel may be present in the liquid fuel composition with the high temperature antioxidant additive in any suitable amount. As previously described, the liquid fuel may include any suitable liquid fuel, including a combination of two or more different fuels. In some embodiments, the liquid fuel may be present in the liquid fuel composition in an amount ranging from 98% to 99.99999% by weight of the liquid fuel composition, or from 99% to 99.99999% by weight of the liquid fuel composition, or from 99% to 99.999999% by weight of the liquid fuel composition. One of ordinary skill in the art, with the benefit of this disclosure, should be able to select an appropriate liquid fuel and amount thereof to include in the liquid fuel composition for a particular application.

In some embodiments, additional fuel additives can be included in the liquid fuel composition as desired by one of ordinary skill in the art for a particular application. Examples of these additional additives include, but are not limited to, detergents, rust inhibitors, corrosion inhibitors, lubricants, antifoaming agents, demulsifiers, conductivity improvers, metal deactivators, cold-flow improvers, cetane improvers, octane improvers and fluidizers, among others. One of ordinary skill in the art, with the benefit of this disclosure, should be able to select additional additives and amounts thereof as needed for a particular application.

In another form of the instant invention, the high temperature antioxidant additive comprising alkylpiperidin-1-yl alkylcarbamate may be included in polymer compositions, lubricating oil compositions or grease compositions in order to improve thermal stability and oxidative stability. Non-limiting exemplary polymer compositions include low density polyethylene, linear low density polyethylene, high density polyethylene, polypropylene, copolymers of ethylene and propylene, terpolymers of ethylene, propylene and butene-1, polybutylene, polyester, polyamides and polyvinyl chloride. In such polymer compositions, the inventive high temperature antioxidant additive comprising alkylpiperidin-1-yl alkylcarbamate provides benefits in such non-limiting exemplary polymer processes as extrusion, injection molding, thermoforming, and blow molding, wherein the polymer composition is subjected to elavated temperatures. In lubricating oil compositions, the high temperature antioxidant additive comprising alkylpiperidin-1-yl alkylcarbamate may be added to one or more base oils in the composition with one or more lubricating additives to enhance thermal and oxidative stability of the lubricating oil. In grease compositions, the high temperature antioxidant additive comprising alkylpiperidin-1-yl alkylcarbamate may be added to one or more base oils, one or more thickeners and/or one or more grease additives to enhance thermal and oxidative stability of the grease.

### EXAMPLES

To facilitate a better understanding of the present invention, the following examples of certain aspects of some embodiments are given. In no way should the following examples be read to limit, or define, the entire scope of the invention.

### Example 1

2,2,6,6-tetramethylpiperidin-1-yl (2-ethylhexyl)carbamate was produced in a dried round bottom flask containing a stir bar charged with 2-ethylhexan-1-amine (1 mmol) and trimethylamine (3 mmol) in dimethylformamide (2.0 mL) and the 0.5 M solution was cooled to 0° C. on an ice-bath before treatment with 4-nitrophenyl (2,2,6,6-tetramethylpiperidin-1-yl) carbonate (1.2 mmol) as a solid in two portions. The reaction mixture turns to an orange suspension. The reaction mixture was warmed to ambient conditions and stirred at room temperature for 16 h. The reaction mixture was quenched by water and extracted with ethyl acetate. The combine organic layer was washed with water and brine (2 times). The organic layer was dried over sodium sulphate and concentrated under reduced pressure to afford the orange semi-solid. The crude product was purified by column chromatography using Ethyl acetate/Heptane (5:95) to obtain off-white solid.

### Example 2

2,2,6,6-tetramethylpiperidin-1-yl octadecylcarbamate was prepared as in Inventive Example 1 except that octadecylamine was used in place of 2-ethylhexan-1-amine.

### Example 3

2,2,6,6-tetramethylpiperidin-1-yl octylcarbamate was prepared as in Inventive Example 1 except that octylamine was used in place of 2-ethylhexan-1-amine.

### Example 4

Additional testing was performed to further evaluate the high temperature antioxidant additives. For this additional testing, an antioxidant additive was added to a ULSD. The antioxidant additive was 2,2,6,6-tetramethylpiperidin-1-yl (2-ethylhexyl)carbamate. The oxidation induction period was then measured for this sample liquid fuel composition using the PetroOXY automatic oxidation stability tester as described above. For comparative purposes, the oxidation induction period for the same ULSD was also tested without the addition of the antioxidant additive. The test temperature was 180° C.

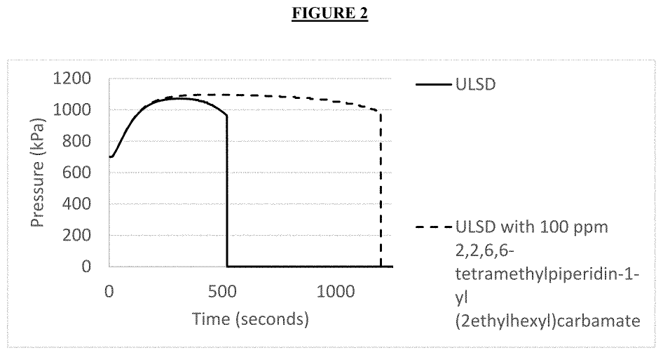

FIG. 2 is a chart showing the oxidation induction period for these tests. As illustrated, the addition of 100 ppm of the high temperature antioxidant additive extended the oxidation induction period for the ULSD by more than 670 seconds. Indeed, the addition of the antioxidant additive extended the oxidation induction period by approximately 131%. The point where there is a precipitous drop in the pressure trace highlights the end of tests and the time where the pressure drop equals 10% from its maximum value. Extending this point to zero pressure in the trace helps delineate the end point in the data where no further data was collected and the heating to the sample was discontinued.

### Example 5

An antioxidant additive was added to a ULSD in an amount of 100 ppm. The antioxidant additive was 2,2,6,6-tetramethylpiperidin-1-yl octyldecylcarbamate. The oxidation induction period was then measured for this sample liquid fuel composition using the PetroOXY automatic oxidation stability tester as described above. For comparative purposes, the oxidation induction period for the same ULSD was also tested without the addition of the antioxidant additive. The test temperature was 180° C.

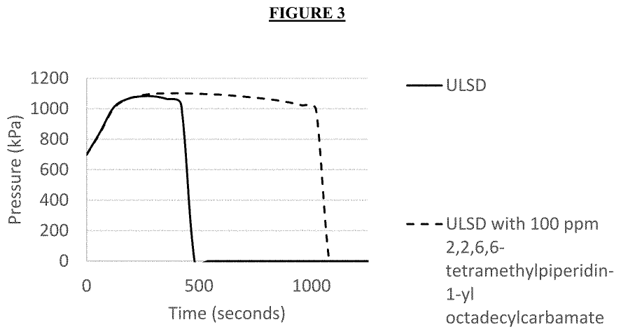

FIG. 3 is a chart showing the oxidation induction period for these tests. As illustrated, the addition of 100 ppm of the high temperature antioxidant additive extended the oxidation induction period for the ULSD by more than 600 seconds. Indeed, the addition of the antioxidant additive extended the oxidation induction period by approximately 89%. The point where there is a precipitous drop in the pressure trace highlights the end of tests and the time where the pressure drop was 10% from its maximum value. Extending this point to zero pressure in the trace helps delineate the end point in the data where no further data was collected and the heating to the sample was discontinued.

### Example 6

Additional testing was performed to further evaluate the high temperature antioxidant additives. For this additional testing, an antioxidant additive was added to a ULSD. The antioxidant additive was 2,2,6,6-tetramethylpiperidin-1-yl octylcarbamate. The oxidation induction period was then measured for this sample liquid fuel composition using the PetroOXY automatic oxidation stability tester as described above. For comparative purposes, the oxidation induction period for the same ULSD was also tested without the addition of the antioxidant additive. The test temperature was 180° C.

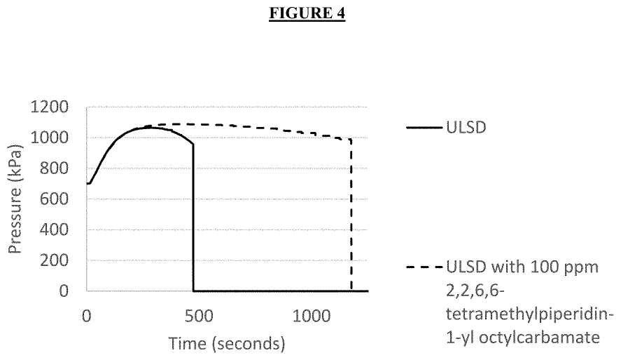

FIG. 4 is a chart showing the oxidation induction period for these tests. As illustrated, the addition of 100 ppm of the high temperature antioxidant additive extended the oxidation induction period for the ULSD by more than 700 seconds. Indeed, the addition of the antioxidant additive extended the oxidation induction period by approximately 149%. The point where there is a precipitous drop in the pressure trace highlights the end of tests and the time where the pressure drop equals 10% from its maximum value. Extending this point to zero pressure in the trace helps delineate the end point in the data where the no further data was collected and the heating to the sample was discontinued.

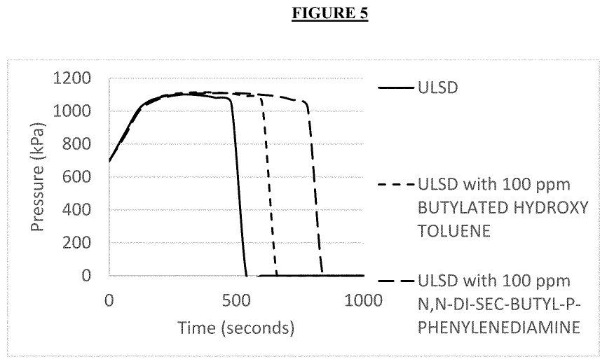

FIG. 5 is a chart showing the oxidation induction period for a ULSD with 100 ppm of known storage stability antioxidant additives. The antioxidant additives used were butylated hydroxytoluene and N,N′-Di-sec-butyl-p-phenylenediamine. As illustrated in the Figure, the addition of 100 ppm of butylated hydroxytoluene, a storage stability antioxidant additive, extended the oxidation induction period for the ULSD by 120 seconds. The addition of the storage stability antioxidant additive extended the oxidation induction period by approximately 22%. Also illustrated, the addition of 100 ppm of N,N′-Di-sec-butyl-p-phenylenediamine, a storage stability antioxidant additive, extended the oxidation induction period for the ULSD by 240 seconds. The addition of the storage stability antioxidant additive extended the oxidation induction period by approximately 44%. The point where there is a precipitous drop in the pressure trace highlights the end of tests and the time where the pressure drop equals 10% from its maximum value. Extending this point to zero pressure in the trace helps delineate the end point in the data where the no further data was collected and the heating to the sample was discontinued.

Table 1 below summarizes the performance benefits of the high temperature antioxidant additives over the storage stability antioxidant additives.

TABLE 1
Change in induction period at 180° C. for ULSD with 100 ppm of the additive

| Additive | Type | % change |
|---|---|---|
| 2,2,6,6-tetramethylpiperidin-1-yl (2ethylhexyl)carbamate | Ex 1 high temperature AO | 131 |
| 2,2,6,6-tetramethylpiperidin-1-yl octadecylcarbamate | Ex 2 high temperature AO | 89 |
| 2,2,6,6-tetramethylpiperidin-1-yl octylcarbamate | Ex 3 high temperature AO | 149 |
| Butylated hydroxytoluene | Storage stability AO | 22 |
| N,N′-Di-sec-butyl-p-phenylenediamine | Storage stability AO | 44 |

AO = antioxidant

While the invention has been described with respect to a number of embodiments and examples, those skilled in the art, having benefit of this disclosure, will appreciate that other embodiments can be devised which do not depart from the scope and spirit of the invention as disclosed herein. Although individual embodiments are discussed, the invention covers all combinations of all those embodiments.

While compositions, methods, and processes are described herein in terms of “comprising,” “containing,” “having,” or “including” various components or steps, the compositions and methods can also “consist essentially of or “consist of” the various components and steps. The phrases, unless otherwise specified, “consists essentially of and “consisting essentially of do not exclude the presence of other steps, elements, or materials, whether or not, specifically mentioned in this specification, so long as such steps, elements, or materials, do not affect the basic and novel characteristics of the invention, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

All patents, test procedures, and other documents cited herein, including priority documents, are fully incorporated by reference to the extent such disclosure is not inconsistent with this invention and for all jurisdictions in which such incorporation is permitted.

PCT/EP Clauses:

1. A composition comprising the following formula:

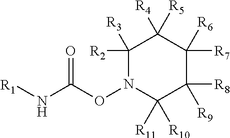

wherein R₁ is a hydrogen, an alkyl group, an alkenyl group, a heteroatom substituted alkyl group, or a heteroatom substituted alkenyl group, and wherein R₂, R₃, R₄, R₅, R₆, R₇, R₈, R₉, R₁₀, and R₁₁are each independently selected from the group consisting of hydrogen, an alkyl group, an alkenyl group, a heteroatom substituted alkyl group, a heteroatom substituted alkenyl group, and a hydroxyl group.

2. The composition of clause 1, wherein the R₂, R₃, R₁₀ and R₁₁ are methyl groups.

3. The composition of clauses 1-2 comprising the following formula:

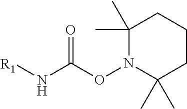

wherein is R₁ is an alkyl group.

4. The composition of clause 3, wherein the alkyl group is a C5 or higher.

5. The composition of clauses 1-4, wherein the composition is used as an antioxidant additive in a liquid fuel composition, a polymer composition, a lubricating oil composition, or a grease composition.

6. A liquid fuel composition comprising: a liquid fuel; and an effective amount of an antioxidant additive comprising the following formula:

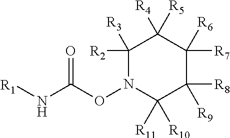

wherein R₁ is a hydrogen, an alkyl group, an alkenyl group, a heteroatom substituted alkyl group, or a heteroatom substituted alkenyl group, and wherein R₂, R₃, R₄, R₅, R₆, R₇, R₈, R₉, R₁₀, and R₁₁ are each independently selected from the group consisting of hydrogen, an alkyl group, an alkenyl group, a heteroatom substituted alkyl group, a heteroatom substituted alkenyl group, and a hydroxyl group.

7. The liquid fuel composition of clause 6, wherein the liquid fuel is selected from the group consisting of a motor gasoline, an aviation fuel, a supersonic fuel, a rocket fuel, a marine fuel, a diesel fuel, and combinations thereof

8. The liquid fuel composition of clauses 6-7, wherein the liquid fuel comprises a mixture of an ultra-low sulfur diesel fuel, and a renewable fuel including a biofuel composition, a biodiesel composition or combinations thereof.

9. The liquid fuel composition of clause 8, wherein the supersonic fuel comprises a mixture selected from the group consisting of alkanes, cycloalkanes, alkylbenzenes, tetralins, and naphthalenes.

10. The liquid fuel composition of clauses 6-9, wherein the liquid fuel comprises greater than or equal to about 98 vol. % of the overall liquid fuel composition.

11. The liquid fuel composition of clauses 6-10, wherein the R₂, R₃, R₁₀ and R₁₁ are methyl groups.

12. The liquid fuel composition of clauses 6-11, wherein the antioxidant additive comprises the following formula:

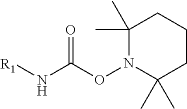

wherein is R₁ is a alkyl group.

13. The liquid fuel composition of clause 12, wherein the alkyl group is a C5 or higher.

14. The liquid fuel composition of clauses 6-13, wherein the effective amount of the antioxidant additive ranges from about 0.1 ppm to about 500 ppm of the overall liquid fuel composition.

15. The liquid fuel composition of clauses 6-14, wherein the liquid fuel is present in an amount of about 99 vol. % or greater of the overall liquid fuel composition, and wherein the effective amount of the antioxidant additive ranges from about 1 ppm to about 100 ppm of the overall liquid fuel composition.

16. The liquid fuel composition of clauses 6-15, further comprising at least one additional additive selected from the group consisting of a detergent, a rust inhibitor, a corrosion inhibitor, a lubricant, an antifoaming agent, a demulsifier, a conductivity improver, a metal deactivator, a cold-flow improver, a cetane improvers, fluidizer, and combinations thereof.

17. A method for improving thermal oxidative stability of a liquid fuel composition comprising: providing a liquid fuel composition to an internal combustion engine, wherein the liquid fuel composition comprises a liquid fuel; and an effective amount of an antioxidant additive comprising the following formula:

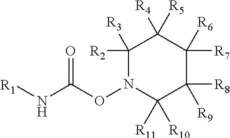

wherein R₁ is a hydrogen, an alkyl group, an alkenyl group, a heteroatom substituted alkyl group, or a heteroatom substituted alkenyl group, and wherein R₂, R₃, R₄, R₅, R₆, R₇, R₈, R₉, R₁₀, and R₁₁ are each independently selected from the group consisting of hydrogen, an alkyl group, an alkenyl group, a heteroatom substituted alkyl group, a heteroatom substituted alkenyl group, and a hydroxyl group, and combusting the liquid fuel composition in the internal combustion engine.

What is claimed is:

1. A composition comprising the following formula: 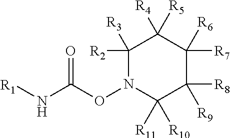
wherein R₁ is a hydrogen, an alkyl group, an alkenyl group, a heteroatom substituted alkyl group, or a heteroatom substituted alkenyl group, and
wherein R₂, R₃, R₄, R₅, R₆, R₇, R₈, R₉, R₁₀, and R₁₁ are each independently selected from the group consisting of hydrogen, an alkyl group, an alkenyl group, a heteroatom substituted alkyl group, a heteroatom substituted alkenyl group, and a hydroxyl group.

2. The composition of claim 1, wherein R₂, R₃, R₁₀ and R₁₁ are methyl groups.

3. The composition of claim 2 comprising the following formula: 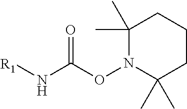
wherein is R₁ is an alkyl group.

4. The composition of claim 3, wherein the alkyl group is a C5 or higher.

5. The composition of claim 1, wherein the composition is used as an antioxidant additive in a liquid fuel composition, a polymer composition, a lubricating oil composition, or a grease composition.

6. A liquid fuel composition comprising:
a liquid fuel; and
an effective amount of an antioxidant additive comprising the following formula: 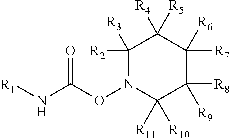
wherein R1 is a hydrogen, an alkyl group, an alkenyl group, a heteroatom substituted alkyl group, or a heteroatom substituted alkenyl group, and
wherein R2, R3, R4, R5, R6, R7, R8, R9, R10, and R11 are each independently selected from the group consisting of hydrogen, an alkyl group, an alkenyl group, a heteroatom substituted alkyl group, a heteroatom substituted alkenyl group, and a hydroxyl group.

7. The liquid fuel composition of claim 6, wherein the liquid fuel is selected from the group consisting of a motor gasoline, an aviation fuel, a supersonic fuel, a rocket fuel, a marine fuel, a diesel fuel, and combinations thereof

8. The liquid fuel composition of claim 6, wherein the liquid fuel comprises a mixture of an ultra-low sulfur diesel fuel, and a renewable fuel including a biofuel composition, a biodiesel composition or combinations thereof

9. The liquid fuel composition of claim 7, wherein the supersonic fuel comprises a mixture selected from the group consisting of alkanes, cycloalkanes, alkylbenzenes, tetralins, and naphthalenes.

10. The liquid fuel composition of claim 6, wherein the liquid fuel comprises greater than or equal to about 98 vol. % of the overall liquid fuel composition.

11. The liquid fuel composition of claim 6, wherein R2, R3, R10 and R11 are methyl groups.

12. The liquid fuel composition of claim 11, wherein the antioxidant additive comprises the following formula: 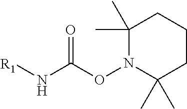
wherein is R₁ is a alkyl group.

13. The liquid fuel composition of claim 12, wherein the alkyl group is a C5 or higher.

14. The liquid fuel composition of claim 6, wherein the effective amount of the antioxidant additive ranges from about 0.1 ppm to about 500 ppm of the overall liquid fuel composition.

15. The liquid fuel composition of claim 6, wherein the liquid fuel is present in an amount of about 99 vol. % or greater of the overall liquid fuel composition, and wherein the effective amount of the antioxidant additive ranges from about 1 ppm to about 100 ppm of the overall liquid fuel composition.

16. The liquid fuel composition of claim 6, further comprising at least one additional additive selected from the group consisting of a detergent, a rust inhibitor, a corrosion inhibitor, a lubricant, an antifoaming agent, a demulsifier, a conductivity improver, a metal deactivator, a cold-flow improver, a cetane improvers, fluidizer, and combinations thereof.

17. A method for improving thermal oxidative stability of a liquid fuel composition comprising:
providing a liquid fuel composition to an internal combustion engine, wherein the liquid fuel composition comprises a liquid fuel; and an effective amount of an antioxidant additive comprising the following formula: 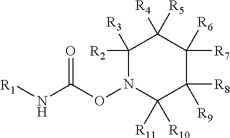
wherein R1 is a hydrogen, an alkyl group, an alkenyl group, a heteroatom substituted alkyl group, or a heteroatom substituted alkenyl group, and
wherein R2, R3, R4, R5, R6, R7, R8, R9, R10, and R11 are each independently selected from the group consisting of hydrogen, an alkyl group, an alkenyl group, a heteroatom substituted alkyl group, a heteroatom substituted alkenyl group, and a hydroxyl group, and combusting the liquid fuel composition in the internal combustion engine. The method of claim 17, wherein the thermal oxidative stability as measured by rapid small scale oxidation test or pressure vessel test is improved by from about 10% to about 200% for the liquid fuel composition compared to a liquid fuel composition not including the effective amount of the antioxidant additive.

18. The method of claim 17, wherein the liquid fuel is selected from the group consisting of a motor gasoline, an aviation gasoline, an aviation turbine fuel, a supersonic fuel, a rocket fuel, a marine fuel, a diesel fuel, and combinations thereof.

19. The method of claim 17, wherein R₁ is an alkyl or alkenyl group ranging from 5 carbon atoms to 18 carbon atoms and wherein R₂, R₃, R₁₀, and R₁₁ are methyl groups and R₄, R₅, R₆, R₇, R₈, R₉ are each hydrogen.

20. The method of claim 17, wherein the internal combustion engine is a direct injection engine.

21. The method of claim 17, wherein the internal combustion engine is a supersonic turbine engine.

22. The method of claim 17, wherein the internal combustion engine is a common-rail direct fuel injection engine.
